(19) United States
(12) Patent Application Publication
(10) Pub. No.: US 2025/0041386 A1
(43) Pub. Date: Feb. 6, 2025

(54) Recombinant Interleukin 12 Construct and Uses Thereof

(71) Applicant: Janssen Sciences Ireland Unlimited Company, Co. Cork (IE)

(72) Inventors: Helen Horton, Beerse (BE); Dorien De Pooter, Beerse (BE); Daniel Boden, Beerse (BE)

(21) Appl. No.: 17/596,538

(22) PCT Filed: Jun. 18, 2020
(86) PCT No.: PCT/IB2020/055705

§ 371 Date: Dec. 13, 2021

(30) Foreign Application Priority Data
Jun. 18, 2019 (EP) 19180939.1

(51) Int. Cl. A61K 38/20; A61P 37/04; C07K 14/54

(52) U.S. Cl. CPC A61K 38/208; A61P 37/04; C07K 14/5434; C07K 2319/02

(57) ABSTRACT

IL12 fusion proteins containing an IL12 p40 subunit, a linker and an IL12 p35 subunit are described. Also described are nucleic acids encoding the fusion proteins, vectors comprising the nucleic acids, and methods of using the fusion proteins and the nucleic acids encoding the fusion proteins to enhance immune responses to antigens.

### CROSS REFERENCE TO RELATED APPLICATION

This application claims priority to European Application No. EP19180939.1 filed on Jun. 18, 2019, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “Sequence Listing” and a creation date of Jun. 10, 2020 and having a size of 43.1 kb. The sequence listing submitted via EFS-Web is part of the specification and is herein incorporated by reference in its entirety.

### FIELD OF THE INVENTION

The invention relates to recombinant IL12 fusion proteins. In particular, the invention relates to a fusion protein comprising an IL12 p40 subunit, a linker and an IL12 p35 subunit, nucleic acids and expression vectors encoding the fusion proteins, recombinant cells thereof and pharmaceutical compositions comprising the fusion proteins. The invention also relates to methods of using the fusion proteins as adjuvants to enhance immune responses to antigens.

### BACKGROUND OF THE INVENTION

Interleukin 12 (IL12) is an interleukin that is naturally produced by dendritic cells, macrophages, neutrophils, and human B-lymphoblastoid cells in response to antigenic stimulation. IL12 is a proinflammatory cytokine that promotes differentiation of naïve CD4 T cells into TH1 helper cells, induces proliferation, induces interferon gamma (IFNg) production by T cells and enhances cytotoxicity of natural killer (NK) and cytotoxic T cells (Trinchieri et al., Nat Rev Immunol. 2003 February; 3(2):133-46). IL12 is a heterodimeric protein encoded by two separate genes, IL12A (p35) and IL12B (p40). When the subunits combine together, they form the functional protein, IL12 p70 (Kobayashi et al., J Exp Med. 1989-9-1; 170(3):827-45).

Several studies have reported that IL12 as a protein has a critical role in inducing antiviral and antitumor effects in vivo. Direct administration of IL12 protein or cDNA expressing IL-12 by gene gun can affect tumor progression and metastases in animal models (Dias et al., Int J Cancer. 1998-1-5; 75(1):151-7; Rakhmilevich et al., Proc Natl Acad Sci USA. 1996-6-25; 93(13):6291-6; Yu et al., J Leukoc Biol. 1997 October; 62(4):450-7). Similarly, therapeutic treatments with IL12 protein can result in protective responses to some infectious viral agents (Bi et al., J Immunol. 1995 Dec. 15; 155(12):5684-9; Orange and Biron. J Immunol. 1996-6-15; 156(12):4746-56).

The clinical development of IL12 as a single agent for systemic cancer therapy has been hindered by its significant toxicity and disappointing anti-tumor effects (Motzer et al., Clin. Cancer Res. 1998; 4:1183-1191; Sangro et al., J. Clin. Oncol. 2004; 22:1389-1397. The lack of efficacy was accompanied by, and probably related to, the declining biological effects of IL12 in the course of repeated administrations at doses approaching the maximum tolerated dose (MTD) (Leonard et al., Blood. 1997; 90:2541-2548; Cohen. Science. 1995-11-10; 270(5238):908). Nevertheless, IL12 remains a very promising immunotherapeutic agent because recent cancer vaccination studies in animal models and humans have demonstrated its powerful adjuvant properties (Portielje et al., Cancer Immunol Immunother. 2003 March; 52(3):133-44).

There is a need for a novel IL12 construct that can be expressed efficiently and used effectively as an immunotherapeutic agent.

### BRIEF SUMMARY OF THE INVENTION

In one general aspect the invention relates to a fusion protein comprising an IL12 p40 subunit, a linker, and an IL12 p35 subunit. In certain embodiments the fusion protein comprises a) an IL12 p40 subunit; b) a linker consisting of the amino acid sequence of SEQ ID NO:3; and c) an IL12 p35 subunit; wherein the fusion protein is arranged from N-terminus to C-terminus in the order (a)-(b)-(c), and the C-terminus of the IL12 p40 subunit is fused to the N-terminus of the IL12 p35 subunit through the linker.

In certain embodiments, the p40 subunit comprises an amino acid sequence having at least 90% sequence identity to SEQ ID NO: 1, 7, or 9. In certain embodiments, the p35 subunit comprises an amino acid sequence having at least 90% sequence identity to SEQ ID NO: 2, 8, or 10.

In certain embodiments the fusion protein comprises an amino acid sequence having at least 90% sequence identity to SEQ ID NO: 24. In certain embodiments the fusion protein comprises an amino acid sequence having at least 90% sequence identity to SEQ ID NO: 25. In certain embodiments the fusion protein comprises an amino acid sequence having at least 90% sequence identity to SEQ ID NO: 26.

In certain embodiment the fusion protein further comprises a signal sequence operably linked to the N-terminus of the p40 subunit. The signal sequence can, for example, be selected from the group consisting of SEQ ID NOs: 11, 12, and 13.

In another general aspect, the invention relates to an isolated nucleic acid molecule comprising a nucleotide sequence encoding the fusion protein of the invention. The isolated nucleic acid molecule can, for example, have at least 90% sequence identity to SEQ ID NOs: 27, 28, 29.

In another general aspect, the invention relates to a vector comprising a nucleic acid molecule encoding a fusion protein of the invention.

In another general aspect, the invention relates to a host cell comprising a nucleic acid molecule encoding a fusion protein of the invention.

In another general aspect, the invention relates to a pharmaceutical composition comprising the fusion protein of the invention and a pharmaceutically acceptable carrier. In certain embodiments the pharmaceutical composition comprises nucleic acid molecules encoding the fusion protein and a pharmaceutically acceptable carrier. In certain embodiments the pharmaceutical composition comprises vectors comprising nucleic acid molecules encoding the fusion protein and a pharmaceutically acceptable carrier.

Also provided are kits or pharmaceutical combinations comprising the fusion proteins, the nucleic acids, or the vectors of the invention and an immunogen. In certain embodiments, the immunogen is capable of inducing an immune response against an infectious agent, or a disease.

Also provided are methods of enhancing an immune response in a subject in need thereof. In certain embodiments, the method comprises administering to the subject an effective amount of the therapeutic composition of the fusion proteins, the nucleic acid molecules, or the vectors of the invention.

Also provided are methods of inducing an immune response in a subject in need thereof. In certain embodiments, the method comprises administering to the subject an effective amount of an immunogen and at least one of the fusion proteins, the nucleic acid molecule, and the vectors of the invention.

Also provided are the fusion protein, the nucleic acid molecule or the vectors of the invention for use in enhancing an immune response in a subject in need thereof.

Also provided are immunogenic combinations or kits comprising an immunogen and at least one of the fusion proteins, the nucleic acid molecule and the vectors of the invention for use in inducing an immune response in a subject in need thereof.

Other aspects of the application include products containing the immunogenic combination or kit of the invention as a combined preparation for simultaneous, separate or sequential use in enhancing an immune response induced by the immunogen, in a subject in need thereof.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a DNA plasmid encoding a human IL12 fusion protein according to an embodiment of the application the IL12 fusion protein is expressed under control of a CMV promoter with a signal sequence located between the CMV promoter and the polynucleotide sequence encoding the fusion protein; transcriptional regulatory elements of the plasmid include a bGH polyadenylation sequence located downstream of the polynucleotide sequence encoding the fusion protein; a second expression cassette is included in the plasmid including an f1 origin, a neomycin resistance gene under the control of an SV40 early promoter, and an SV40 polyadenylation sequence; a third expression cassette is included in the plasmid in reverse orientation including a ampicillin resistance gene under control of an Amp^{r}(bla) promoter; an origin of replication (pUC) is also included in reverse orientation.

FIG. 2 shows a DNA plasmid encoding a human IL12 fusion protein according to an embodiment of the application the IL12 fusion protein is expressed under control of a CMV promoter with a signal sequence located between the CMV promoter and the polynucleotide sequence encoding the fusion protein; transcriptional regulatory elements of the plasmid include an enhancer sequence located between the CMV promoter and the polynucleotide sequence encoding the fusion protein and a bGH polyadenylation sequence located downstream of the polynucleotide sequence encoding the fusion protein; a second expression cassette is included in the plasmid in reverse orientation including a kanamycin resistance gene under control of an Amp^{r}(bla) promoter; an origin of replication (pUC) is also included in reverse orientation.

FIG. 3 shows ELISA measurements of IL12 p70 concentrations from the media supernatant of HEK293T cells transfected with pcDNA-p35 and pcDNA-p40 expressing plasmids or pcDNA plasmids expressing fusion proteins with KE, FA, IR, and TC linkers located between the p40 and p35 subunits according to embodiments of the application; the IL12 p70 concentration is indicated on the x-axis expressed as pg/ml.

FIG. 4 shows is a Western blot analysis showing a comparison of p40 expression in HEK293T cells transfected with either p40 and p35 expressing plasmids or p40-KE-p35 fusion protein expressing plasmid; lane 1: pcDNA-p40-KE-p35 fusion construct cell lysate; lane 2: pcDNA-p40 and pcDNA-p35 cell lysate; lane 3: empty; lane 4: pcDNA-p40-KE-p35 media supernatant; lane 5: pcDNA-p40 and pcDNA-p35 media supernatant.

FIG. 5 shows interferon gamma (IFNγ) protein expression in the media supernatant of two human CD3 T cell samples (DN921 and DN922) after stimulation with increasing concentrations of recombinant IL12 p70 or the supernatant of HEK293T cells transfected with either p40 and p35 expressing plasmids or p40-KE-p35 fusion protein expressing plasmid; IFNγ concentration is indicated on the y-axis expressed as pg/ml; the IL12 p70 concentration is indicated on the x-axis expressed as pg/ml.

FIG. 6 shows a DNA plasmid encoding a mouse IL12 fusion protein according to an embodiment of the application; the IL12 fusion protein is expressed under control of a CMV promoter with a signal sequence located between the CMV promoter and the polynucleotide sequence encoding the fusion protein and a SV40 polyadenylation sequence located downstream of the polynucleotide sequence encoding the fusion protein; a second expression cassette is included in the plasmid in reverse orientation including an ampicillin resistance gene under control of an Amp^{r} (bla) promoter; an origin of replication (pUC) is also included in reverse orientation.

FIG. 7 shows ELISPOT responses of Balb/C mice immunized with a combination of DNA plasmids expressing an IL12 fusion protein and HBV antigens according the study described in Example 4; Group 1, single Core and Pol pDNA; Group 2, Core and Pol pDNA and 0.1 ug mIL12 fusion protein pDNA; Group 3, Core and Pol pDNA and 0.5 ug mIL12 fusion protein pDNA; Group 4, Core and Pol pDNA and 2 ug mIL12 fusion protein pDNA; Group 5, Core and Pol pDNA and 0.1 ug pUMVC3 mIL12-IRES (Ichor) pDNA; Group 6, Core and Pol pDNA and 0.5 ug pUMVC3 mIL12-IRES (Ichor) pDNA; Group 7, Core and Pol pDNA and 2 ug pUMVC3 mIL12-IRES (Ichor) pDNA; Group 8, Empty pDK vector; peptide pools used to stimulate splenocytes isolated from the various vaccinated animal groups are indicated in gray scale; the number of responsive T-cells are indicated on the y-axis expressed as spot forming cells (SFC) per 10⁶ splenocytes.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Fusion Proteins

The invention generally relates to a fusion protein comprising an IL12 p40 subunit, a linker, and an IL12 p35 subunit.

As used herein, the term “fusion protein” refers to a protein having two or more portions covalently linked together, where each of the portions is derived from different proteins.

As used herein, the terms “IL12” and “IL12 p70” and “NK cell stimulatory factor (NKSF)” are used interchangeably and refer to the interleukin 12 protein. IL12 p70 is a heterodimeric protein encoded by two separate genes, IL12A (p35) and IL12B (p40). As used herein, the terms “IL12A,” “IL12 p35,” and “p35” are used interchangeably and refer to IL12 subunit alpha protein. As used herein, the terms “IL12B,” “IL12 p40,” and “p40” are used interchangeably and refer to IL12 subunit beta protein.

A suitable linker is used in fusion proteins according to embodiments of the invention. As used herein, the term “linker” refers to a linking moiety comprising a peptide linker. Preferably, the linker helps insure correct folding, minimizes steric hindrance and does not interfere significantly with the structure of each functional component within the fusion protein.

In a general aspect, the invention relates to a fusion protein comprising a) an IL12 p40 subunit; b) a linker consisting of the amino acid sequence of SEQ ID NO: 3; and c) an IL12 p35 subunit; wherein the fusion protein is arranged from N-terminus to C-terminus in the order (a)-(b)-(c), and the C-terminus of the IL12 p40 subunit is fused to the N-terminus of the IL12 p35 subunit through the linker. In another aspect, the invention relates to a fusion protein comprising a) an IL12 p40 subunit; b) a linker consisting of the amino acid sequence of SEQ ID NO: 3; and c) an IL12 p35 subunit; wherein the fusion protein is arranged from N-terminus to C-terminus in the order (c)-(b)-(a), and the C-terminus of the IL12 p35 subunit is fused to the N-terminus of the IL12 p40 subunit through the linker. The IL12 subunits can be from any mammal, such as a human or another suitable mammal, such as a mouse, rabbit, rat, pig, dog, or a primate. In certain embodiments, the p40 subunit comprises an amino acid sequence having at least 90% (e.g., at least 91%, 92%, 93%, 94%, 95%, 96%, 97%, 98%, 99% or 100%) identity to the amino acid sequence of SEQ ID NO: 1, 7, or 9. In certain embodiments, the p35 subunit comprises an amino acid sequence having at least 90% (e.g., at least 91%, 92%, 93%, 94%, 95%, 96%, 97%, 98%, 99% or 100%) identity to the amino acid sequence identity to SEQ ID NO: 2, 8, or 10.

In an embodiment of the application, the fusion protein comprises an amino acid sequence having at least 90% (e.g., at least 91%, 92%, 93%, 94%, 95%, 96%, 97%, 98%, 99% or 100%) identity to the amino acid sequence of SEQ ID NO: 24. In certain embodiments, the fusion protein comprises an amino acid sequence having at least 90% (e.g., at least 91%, 92%, 93%, 94%, 95%, 96%, 97%, 98%, 99% or 100%) identity to the amino acid sequence of SEQ ID NO: 25. In certain embodiments, the fusion protein comprises an amino acid sequence having at least 90% (e.g., at least 91%, 92%, 93%, 94%, 95%, 96%, 97%, 98%, 99% or 100%) identity to the amino acid sequence of SEQ ID NO: 26.

In an embodiment of the application, the fusion protein further comprises a signal sequence operably linked to the N-terminus of the p40 subunit. As used herein, the term “signal sequence” refers to a sequence encoding a signal peptide that targets proteins for secretion and direct transport across the endoplasmic reticulum (ER) membrane. Any signal sequence known to those skilled in the art in view of the present disclosure can be used in the fusion protein of the invention. In preferred embodiments, the signal sequence is selected from the group consisting of SEQ ID NOs: 11, 12, and 13.

### Polynucleotides and Vectors

In another general aspect, the application provides a non-naturally occurring nucleic acid molecule encoding an IL12 fusion protein according to the application, and vectors comprising the non-naturally occurring nucleic acid. The non-naturally occurring nucleic acid molecule can comprise any polynucleotide sequence encoding an IL12 fusion protein of the application, which can be made using methods known in the art in view of the present disclosure. A polynucleotide can be in the form of RNA or in the form of DNA obtained by recombinant techniques (e.g., cloning) or produced synthetically (e.g., chemical synthesis). The DNA can be single-stranded or double-stranded, or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles. In one preferred embodiment, the non-naturally occurring nucleic acid molecule is a DNA molecule. In another preferred embodiment, the non-naturally occurring nucleic acid molecule is a RNA molecule.

In an embodiment of the application, a non-naturally occurring nucleic acid molecule comprises a first polynucleotide sequence encoding a fusion protein consisting of an amino acid sequence that is at least 90% identical to SEQ ID NO: 24, SEQ ID NO:25 or SEQ ID NO:26, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 24, SEQ ID NO:25 or SEQ ID NO:26, preferably 98%, 99% or 100% identical to SEQ ID NO: 24, SEQ ID NO:25 or SEQ ID NO:26. In a particular embodiment of the application, a first non-naturally occurring nucleic acid molecule encodes a fusion protein consisting the amino acid sequence of SEQ ID NO: 24, SEQ ID NO:25 or SEQ ID NO:26.

Examples of polynucleotide sequences of the application encoding an IL12 fusion protein comprising the amino acid sequence of SEQ ID NO: 24, SEQ ID NO:25 or SEQ ID NO:26 include, but are not limited to, a polynucleotide sequence at least 90% identical to SEQ ID NO: 27, SEQ ID NO:28 or SEQ ID NO:29, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 27, SEQ ID NO:28 or SEQ ID NO:29, preferably 98%, 99% or 100% identical to SEQ ID NO: 27, SEQ ID NO:28 or SEQ ID NO:29. Exemplary non-naturally occurring nucleic acid molecules encoding an IL12 fusion protein have the polynucleotide sequence of SEQ ID NO: 27, SEQ ID NO:28 or SEQ ID NO:29.

The nucleic acids of the invention can, for example, be comprised in a vector. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. A vector can be a DNA vector or an RNA vector. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (e.g., mRNA) into the host cell or organism. In the context of the disclosure, this term encompasses promoters, enhancers and other expression control elements (e.g., polyadenylation signals and elements that affect mRNA stability).

In some embodiments of the application, a vector is a non-viral vector. Examples of non-viral DNA vectors include, but are not limited to, DNA plasmids, bacterial artificial chromosomes, yeast artificial chromosomes, closed linear deoxyribonucleic acid, e.g., a linear covalently closed DNA, e.g., a linear covalently closed double stranded DNA molecule, etc. Examples of non-viral RNA vectors include, but are not limited to, RNA replicon, mRNA replicon, modified mRNA replicon or self-amplifying mRNA. Preferably, a non-viral vector is a DNA plasmid.

A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3TM (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3TM, pVAX, pVAX-1, ADVAX, NTC8454, etc. Preferably, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which comprise an arenavirus genomic segment wherein one open reading frame of the genomic segment is deleted or functionally inactivated, arenavirus such as lymphocytic choriomeningitidis virus (LCMV), e.g., clone 13 strain or MP strain, and arenavirus such as Junin virus e.g., Candid #1 strain, etc. The vector can also be a non-viral vector.

Preferably, a viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53.

The vector can also be a linear covalently closed double-stranded DNA vector. As used herein, a “linear covalently closed double-stranded DNA vector” refers to a closed linear deoxyribonucleic acid (DNA) that is structurally distinct from a plasmid DNA. It has many of the advantages of plasmid DNA as well as a minimal cassette size similar to RNA strategies. For example, it can be a vector cassette generally comprising an encoded antigenic sequence, a promoter, a polyadenylation sequence, and telomeric ends. The plasmid-free construct can be synthesized through an enzymatic process without the need for bacterial sequences. Examples of suitable linear covalently closed DNA vectors include, but are not limited to, commercially available expression vectors such as “Doggybone™ closed linear DNA” (dbDNA™) (Touchlight Genetics Ltd.; London, England). See, e.g., Scott et al, Hum Vaccin Immunother. 2015 August; 11(8): 1972-1982, the entire content of which is incorporated herein by reference. Some examples of linear covalently closed double-stranded DNA vectors, compositions and methods to create and use such vectors for delivering DNA molecules, such as active molecules of this invention, are described in US2012/0282283, US2013/0216562, and US2018/0037943, the relevant content of each of which is hereby incorporated by reference in its entirety.

A recombinant vector useful for the application can be prepared using methods known in the art in view of the present disclosure. For example, in view of the degeneracy of the genetic code, several nucleic acid sequences can be designed that encode the same polypeptide. A polynucleotide encoding an IL12 fusion protein of the application can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A vector of the application, e.g., a DNA plasmid, a viral vector (particularly an adenoviral vector), an RNA vector (such as a self-replicating RNA replicon), or a linear covalently closed double-stranded DNA vector, can comprise any regulatory elements to establish conventional function(s) of the vector, including but not limited to replication and expression of the IL12 fusion protein encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., IL12 fusion protein) from a start codon to a stop codon. Regulatory elements of the expression cassette can be operably linked to a polynucleotide sequence encoding an IL12 fusion protein of interest. As used herein, the term “operably linked” is to be taken in its broadest reasonable context, and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an IL12 fusion protein. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the nucleic acid encoding an IL12 fusion protein within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic. Preferably, a promoter is a strong eukaryotic promoter, preferably a cytomegalovirus immediate early (CMV-IE) promoter. A nucleotide sequence of an exemplary CMV-IE promoter is shown in SEQ ID NO:

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an IL12 fusion construct) within an expression cassette of the vector. Enhancer sequences are regulatory DNA sequences that, when bound by transcription factors, enhance the transcription of an associated gene. An enhancer sequence is preferably located upstream of the polynucleotide sequence encoding an IL12 fusion protein, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadeylation signal or a SV40 polyadenylation signal. A nucleotide sequence of an exemplary bGH polyadenylation signal is shown in SEQ ID NO: 19.

Any enhancer sequence known to those skilled in the art in view of the present disclosure can be used. For example, an enhancer sequence can be a human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an IL12 fusion protein, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC. An exemplary nucleotide sequence of a pUC ORI is shown in SEQ ID NO: 21.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., IL12 fusion protein. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kanr), ampicillin resistance gene (Ampr), and tetracycline resistance gene (Tetr), as well as genes conferring resistance to chloramphenicol, bleomycin, spectinomycin, carbenicillin, etc.

Preferably, an antibiotic resistance gene in the antibiotic expression cassette of a vector is a kanamycin resistance gene (Kanr). The sequence of Kanr gene is shown in SEQ ID NO: 23. Preferably, the Kanr gene is codon optimized. An exemplary nucleic acid sequence of a codon optimized Kanr gene is shown in SEQ ID NO: 22. The Kanr can be operably linked to its native promoter, or the Kanr gene can be linked to a heterologous promoter. In a particular embodiment, the Kanr gene is operably linked to the ampicillin resistance gene (Ampr) promoter, known as the bla promoter. An exemplary nucleotide sequence of a bla promoter is shown in SEQ ID NO: 20.

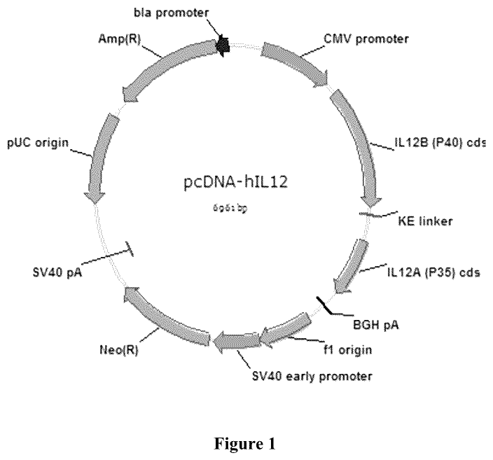

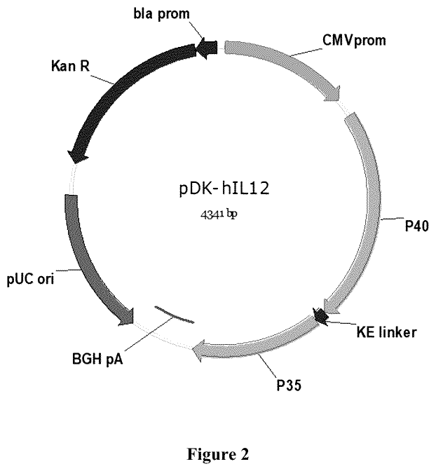

In a particular embodiment of the application, a vector is a DNA plasmid comprising an expression cassette including a polynucleotide encoding an IL12 fusion protein comprising an amino acid sequence at least 90% identical to SEQ ID NO: 24; an upstream sequence operably linked to the polynucleotide encoding the IL12 fusion protein comprising, from 5′ end to 3′ end, a promoter sequence, preferably a CMV promoter sequence of SEQ ID NO: 18; and a downstream sequence operably linked to the polynucleotide encoding the IL12 fusion protein comprising a polyadenylation signal, preferably a bGH polyadenylation signal of SEQ ID NO: 19. Such vector further comprises an antibiotic resistance expression cassette including a polynucleotide encoding an antibiotic resistance gene, preferably a Kanr gene, more preferably a codon optimized Kanr gene that is at least 90% identical to SEQ ID NO: 23, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 23, preferably 100% identical to SEQ ID NO: 23, operably linked to an Ampr (bla) promoter of SEQ ID NO: 20, upstream of and operably linked to the polynucleotide encoding the antibiotic resistance gene; and an origin of replication, preferably a pUC on of SEQ ID NO: 21. Preferably, the antibiotic resistance cassette and the origin of replication are present in the plasmid in the reverse orientation relative to the IL12 fusion protein expression cassette. Exemplary DNA plasmids comprising the above mentioned features are shown in FIG. 1 and FIG. 2.

In another embodiment, a vector is a DNA plasmid comprising an expression cassette including a polynucleotide encoding an IL12 fusion protein comprising an amino acid sequence at least 90% identical to SEQ ID NO: 26; an upstream sequence operably linked to the polynucleotide encoding the IL12 fusion protein comprising, from 5′ end to 3′ end, a promoter sequence, preferably a CMV promoter sequence of SEQ ID NO: 18, and an enhancer sequence; and a downstream sequence operably linked to the polynucleotide encoding the IL12 fusion protein comprising a polyadenylation signal, preferably a bGH polyadenylation signal of SEQ ID NO: 19. Such vector further comprises an antibiotic resistance expression cassette including a polynucleotide encoding an antibiotic resistance gene, preferably a Kanr gene, more preferably a codon optimized Kanr gene that is at least 90% identical to SEQ ID NO: 22, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 22, preferably 100% identical to SEQ ID NO: 22, operably linked to an Ampr (bla) promoter of SEQ ID NO: 20, upstream of and operably linked to the polynucleotide encoding the antibiotic resistance gene; and an origin of replication, preferably a pUC on of SEQ ID NO: 21. Preferably, the antibiotic resistance cassette and the origin of replication are present in the plasmid in the reverse orientation relative to the IL12 fusion protein expression cassette.

The polynucleotides and expression vectors encoding the IL12 fusion proteins of the application can be made by any method known in the art in view of the present disclosure. For example, a polynucleotide encoding an fusion protein can be introduced or “cloned” into an expression vector using standard molecular biology techniques, e.g., polymerase chain reaction (PCR), etc., which are well known to those skilled in the art.

### Cells

The application also provides cells, preferably isolated cells, comprising any of the polynucleotides and vectors described herein. The cells can, for instance, be used for recombinant protein production, or for the production of viral particles.

The nucleic acids of the invention can, for example, be comprised in a host cell. Any cell known to those skilled in the art in view of the present disclosure can be used as a host cell for the isolated nucleic acid molecule of the invention. The cell can, for example, be a mammalian cell. Examples of mammalian host cells are human embryonic kidney 293T (HEK293T) cell, a HEK293F cell, a HeLa cell, a Chinese hamster ovary (CHO) cell, a NIH 3T3 cell, a MCF-7 cell, a Hep G2 cell, a baby hamster kidney (BHK) cell, and a Cos7 cell.

### Compositions and Combinations

The application also relates to compositions, pharmaceutical combinations, more particularly kits, and vaccines comprising fusion proteins, polynucleotides, and/or vectors encoding fusion proteins according to the application. Any of the fusion proteins, polynucleotides, and/or vectors of the application described herein can be used in the compositions, pharmaceutical combinations or kits, and vaccines of the application.

The application provides a pharmaceutical composition comprising a fusion protein of the invention and a pharmaceutically acceptable carrier.

In an embodiment of the application, a pharmaceutical composition comprises a fusion protein comprising a) an IL12 p40 subunit; b) a linker consisting of the amino acid sequence of SEQ ID NO: 3; and c) an IL12 p35 subunit; wherein the fusion protein is arranged from N-terminus to C-terminus in the order (a)-(b)-(c), and the C-terminus of the IL12 p40 subunit is fused to the N-terminus of the IL12 p35 subunit through the linker.

In an embodiment of the application, a pharmaceutical composition comprises a fusion protein comprising an amino acid sequence having at least 90% sequence identity to SEQ ID NOs: 24, 25, or 26; and a pharmaceutically acceptable carrier. In a preferred embodiment, the pharmaceutical composition comprises a fusion protein comprising an amino acid sequence having at least 90% sequence identity to SEQ ID NO: 24; preferably 100% sequence identity to SEQ ID NO: 24.

In an embodiment of the application, a pharmaceutical composition comprises an isolated nucleic acid molecule comprising a nucleotide sequence encoding a fusion protein of the invention and a pharmaceutically acceptable carrier.

In an embodiment of the application, a pharmaceutical composition comprises an isolated nucleic acid molecule having at least 90% sequence identity to SEQ ID NOs: 27, 28, or 29. In a preferred embodiment, the pharmaceutical composition comprises an isolated nucleic acid molecule having at least 90% sequence identity to SEQ ID NO: 27; preferably 100% sequence identity to SEQ ID NO: 27.

In an embodiment of the application, a pharmaceutical composition comprises a vector, such as a DNA plasmid, a viral vector (particularly an adenoviral vector), an RNA vector (such as a self-replicating RNA replicon), or a linear covalently closed double-stranded DNA vector, comprising an isolated nucleic acid molecule encoding a fusion protein comprising an amino acid sequence having at least 90% sequence identity to SEQ ID NOs: 24, 25, or 26. In a particular embodiment, the vector comprises an isolated nucleic molecule having at least 90% sequence identity to SEQ ID NOs: 27, 28, or 29. In a preferred embodiment, the vector comprises an isolated nucleic molecule comprising the nucleotide sequence of SEQ ID NO: 27.

In another general aspect, the invention relates to a pharmaceutical combination or a kit comprising the fusion protein according to embodiments of the invention and another immunogen. Any of the fusion proteins, polynucleotides, and/or vectors of the application described herein can be used in the pharmaceutical combinations or kits of the invention. Any immunogen of interest can be used in the pharmaceutical combinations or kits of the invention. The pharmaceutical combination can be formulated in one pharmaceutical composition or separate compositions. In an embodiment of the application, a pharmaceutical combination or kit comprises the fusion protein comprising an amino acid sequence having at least 90% sequence identity to SEQ ID NO: 24, 25, or 26 and another immunogen. In a preferred embodiment, the pharmaceutical combination or kit comprises a fusion protein comprising an amino acid sequence having at least 90% sequence identity to SEQ ID NO: 24; preferably 100% sequence identity to SEQ ID NO: 24, and another immunogen.

In an embodiment of the application, a pharmaceutical combination or kit comprises an isolated nucleic acid molecule comprising a nucleotide sequence encoding a fusion protein of the invention and an immunogen. In an embodiment of the application, a pharmaceutical combination or kit comprises an isolated nucleic acid molecule having at least 90% sequence identity to SEQ ID NOs: 27, 28, or 29 and an immunogen. In a preferred embodiment, the pharmaceutical composition comprises an isolated nucleic acid molecule having at least 90% sequence identity to SEQ ID NO: 27; preferably 100% sequence identity to SEQ ID NO: 27 and an immunogen.

In an embodiment of the application, a pharmaceutical combination or kit comprises an immunogen and a vector, such as a DNA plasmid, a viral vector (particularly an adenoviral vector), an RNA vector (such as a self-replicating RNA replicon), or a linear covalently closed double-stranded DNA vector, comprising an isolated nucleic acid molecule encoding a fusion protein comprising an amino acid sequence having at least 90% sequence identity to SEQ ID NOs: 24, 25, or 26. In a particular embodiment, the vector comprises an isolated nucleic molecule having at least 90% sequence identity to SEQ ID NOs: 27, 28, or 29. In a preferred embodiment, the vector comprises an isolated nucleic molecule comprising the nucleotide sequence of SEQ ID NO: 27.

Compositions and combinations of the application can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. Pharmaceutically acceptable carriers can include vehicles, such as lipid nanoparticles (LNPs). The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions and combinations of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, compositions and combinations of the application are formulated for parental injection, preferably subcutaneous, intradermal injection, or intramuscular injection, more preferably intramuscular injection.

According to embodiments of the application, compositions and immunogenic combinations for administration will typically comprise a buffered solution in a pharmaceutically acceptable carrier, e.g., an aqueous carrier such as buffered saline and the like, e.g., phosphate buffered saline (PBS). The compositions and immunogenic combinations can also contain pharmaceutically acceptable substances as required to approximate physiological conditions such as pH adjusting and buffering agents. For example, a composition or immunogenic combination of the application comprising plasmid DNA can contain phosphate buffered saline (PBS) as the pharmaceutically acceptable carrier. The plasmid DNA can be present in a concentration of, e.g., 0.5 mg/mL to 5 mg/mL, such as 0.5 mg/mL 1, mg/mL, 2 mg/mL, 3 mg/mL, 4 mg/mL, or 5 mg/mL, preferably at 1 mg/mL.

Compositions and combinations of the application can be formulated as a vaccine according to methods well known in the art. Such compositions can include adjuvants to enhance immune responses. The optimal ratios of each component in the formulation can be determined by techniques well known to those skilled in the art in view of the present disclosure.

In a particular embodiment of the application, a composition or immunogenic combination is a DNA vaccine. DNA vaccines typically comprise bacterial plasmids containing a polynucleotide encoding an antigen of interest under control of a strong eukaryotic promoter. Once the plasmids are delivered to the cell cytoplasm of the host, the encoded antigen is produced and processed endogenously. The resulting antigen typically induces both humoral and cell-medicated immune responses. DNA vaccines are advantageous at least because they offer improved safety, are temperature stable, can be easily adapted to express antigenic variants, and are simple to produce. Any of the DNA plasmids of the application can be used to prepare such a DNA vaccine.

The application also provides methods of making compositions and immunogenic combinations of the application. A method of producing a composition or immunogenic combination comprises mixing an isolated polynucleotide encoding a fusion protein, vector, and/or polypeptide of the application with one or more pharmaceutically acceptable carriers. One of ordinary skill in the art will be familiar with conventional techniques used to prepare such compositions.

### Methods of Inducing an Immune Response

The application also provides methods of inducing or enhancing an immune response in a subject in need thereof, comprising administering to the subject an effective amount of fusion proteins, polynucleotides, and/or vectors encoding fusion proteins according to the application. In certain embodiments, the method further comprises administering to the subject an immunogen. Preferably, the effective amount of fusion proteins, polynucleotides, and/or vectors encoding fusion proteins according to the application is administered in combination with an immunogenically effective amount of immunogen.

As used herein, the terms and phrases “in combination,” “in combination with,” “co-delivery,” and “administered together with” in the context of the administration of two or more therapies or components to a subject refers to simultaneous administration of two or more therapies or components, such as a viral expression vector and an isolated antigenic polypeptide. “Simultaneous administration” can be administration of the two components at least within the same day. When two components are “administered together with” or “administered in combination with,” they can be administered in separate compositions sequentially within a short time period, such as 24, 20, 16, 12, 8 or 4 hours, or within 1 hour, or within 30 minutes, or within 10 minutes, or within 5 minutes, or within 2 minutes, or they can be administered in a single composition at the same time. In the typical embodiment, two components or therapies are administered in separate compositions. The use of the term “in combination with” does not restrict the order in which therapies or components are administered to a subject. For example, a first therapy or component (e.g. viral expression vector) can be administered prior to (e.g., 5 minutes to one hour before), concomitantly with or simultaneously with, or subsequent to (e.g., 5 minutes to one hour after) the administration of a second therapy (e.g., isolated HIV antigenic polypeptide).

As used herein, “an immunogenically effective amount” or “immunologically effective amount” means an amount of a composition or vector sufficient to induce a desired immune effect or immune response in a subject in need thereof. In one embodiment, an immunogenically effective amount means an amount sufficient to induce an immune response in a subject in need thereof, preferably a safe and effective immune response in a human subject in need thereof. In another embodiment, an immunogenically effective amount means an amount sufficient to produce immunity in a subject in need thereof, e.g., provide a therapeutic effect against a disease such as HIV infection or a cancer. An immunogenically effective amount can vary depending upon a variety of factors, such as the physical condition of the subject, age, weight, health, etc. An immunogenically effective amount can readily be determined by one of ordinary skill in the art in view of the present disclosure.

An immunogenically effective amount can be administered in a single step (such as a single injection), or multiple steps (such as multiple injections), or in a single composition or multiple compositions. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

Any of the fusion proteins, polynucleotides, and/or vectors of the application described herein can be used in methods of the application.

As used herein, the terms “immunogen” or “antigen” refers to any agent or substance that can induce an immune response in a subject upon administration.

In certain embodiments, the immunogen is a polypeptide that can bind specifically to a component of the immune system, such as an antibody or a lymphocyte.

In certain embodiments, the immunogen or antigen is encoded by a nucleic acid molecule that may be incorporated into, for example, a polynucleotide or vector of the invention, for subsequent expression of the immunogen or antigen (e.g., a gene product of interest, or fragment thereof (e.g., a polypeptide)). In certain embodiments, the immunogen is capable of inducing an immune response against an infectious agent or disease. The infectious agent can be, but is not limited to, a virus (e.g., human immunodeficiency virus (HV), influenza, respiratory syncytial virus (RSV), Ebola virus, hepatitis B virus HBV), hepatitis C virus (HCV), human papilloma virus (HPV), Epstein-Barr virus, yellow fever virus, rubella virus, varicella zoster virus, variola virus, mumps virus, measles virus, herpes virus and vaccinia virus) or a pathogen (e.g. a bacterial, parasitic, or fungal pathogen). The disease can be, but is not limited to, an oncogenic disease (e.g., melanoma, lung cancer, squamous carcinomas of the lung, head and neck cancer, breast cancer, ovarian cancer, uterine cancer, prostate cancer, gastric carcinoma, cervical cancer, esophageal, carcinoma, bladder cancer, kidney cancer, brain cancer, liver cancer, colon cancer, bone cancer, pancreatic cancer, skin cancer, cutaneous or intraocular malignant melanoma, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, esophagus cancer, small intestine cancer, endocrine system cancer, thyroid gland cancer, parathyroid gland cancer, adrenal gland cancer, sarcoma of soft tissue, urethra cancer, penis cancer, chronic or acute leukemias including acute myeloid leukemia, chronic myeloid leukemia, acute lymphoblastic leukemia, chronic lymphocytic leukemia, solid tumors of childhood, lymphocytic lymphoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, and T-cell lymphoma).

As used herein, “subject” means any animal, particularly a mammal, most particularly a human, who will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more particularly a human.

The phrase “inducing an immune response” when used with reference to the methods described herein encompasses causing a desired immune response or effect in a subject in need thereof against an infectious agent, e.g., HBV. As used herein, the term “therapeutic immunity” or “therapeutic immune response” means that the vaccinated subject is able to control an infection with the pathogenic agent against which the vaccination was done. In an embodiment, “inducing an immune response” means producing an immunity in a subject in need thereof, e.g., to provide a therapeutic effect against a disease, such as cancer. In certain embodiments, “inducing an immune response” refers to causing or improving cellular immunity, e.g., T cell response. In certain embodiments, “inducing an immune response” refers to causing or improving a humoral immune response against an infectious agent or disease. In certain embodiments, “inducing an immune response” refers to causing or improving a cellular and a humoral immune response against an infectious agent or disease.

As used herein, the term “enhancing” when used with respect to an immune response, such as a T cell response, an antibody response, or a NK cell response, refers to an increase in the immune response in a human subject administered with fusion proteins, nucleic acids, and/or vectors of the invention, relative to the corresponding immune response observed from the human subject administered with an immunogen or antigen according to the application alone.

The term “adjuvant” is defined as one or more substances that cause stimulation of the immune system. In this context, an adjuvant is used to enhance an immune response to immunogens or antigens according to the application.

As used herein, “an effective amount” or “immunologically effective amount” means an amount of a composition, polynucleotide, vector, or fusion protein sufficient to induce a desired immune effect or immune response in a subject in need thereof. An effective amount can be an amount sufficient to induce an immune response in a subject in need thereof. An effective amount can be an amount sufficient to produce immunity in a subject in need thereof, e.g., provide a therapeutic effect against a disease. An effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. An effective amount can readily be determined by one of ordinary skill in the art in view of the present disclosure.

As general guidance, an effective amount when used with reference to a DNA plasmid can range from about 0.1 mg/mL to 10 mg/mL of DNA plasmid total, such as 0.1 mg/mL, 0.25 mg/mL, 0.5 mg/mL. 0.75 mg/mL 1 mg/mL, 1.5 mg/mL, 2 mg/mL, 3 mg/mL, 4 mg/mL, 5 mg/mL, 6 mg/mL, 7 mg/mL, 8 mg/mL, 9 mg/mL, or 10 mg/mL. Preferably, an effective amount of DNA plasmid is less than 8 mg/mL, more preferably less than 6 mg/mL, even more preferably 3-4 mg/mL. An immunogenically effective amount can be from one vector or plasmid, or from multiple vectors or plasmids. An effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an effective amount. For example, when two DNA plasmids are used, an effective amount can be 3-4 mg/mL, with 1.5-2 mg/mL of each plasmid. It is also possible to administer an effective amount to a subject, and subsequently administer another dose of an effective amount to the same subject, in a so-called prime-boost regimen. This general concept of a prime-boost regimen is well known to the skilled person in the vaccine field. Further booster administrations can optionally be added to the regimen, as needed.

An immunogenic combination comprising two DNA plasmids, e.g., a first DNA plasmid encoding an IL12 fusion protein and second DNA plasmid encoding another immunogen can be administered to a subject by mixing both plasmids and delivering the mixture to a single anatomic site. Alternatively, two separate immunizations each delivering a single expression plasmid can be performed. In such embodiments, whether both plasmids are administered in a single immunization as a mixture or in two separate immunizations, the first DNA plasmid and the second DNA plasmid can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second DNA plasmids are administered in a ratio of 1:1, by weight.

### Methods of Delivery

Compositions and pharmaceutical combinations of the invention can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, compositions and pharmaceutical combinations are administered parenterally (e.g., by intramuscular injection or intradermal injection) or transdermally.

In some embodiments of the invention in which a composition or combination comprises one or more viral vectors, administration can be by injection through the skin, e.g., intramuscular or intradermal injection, preferably intramuscular injection. Intramuscular injection can be combined with electroporation, i.e., application of an electric field to facilitate delivery of the DNA plasmids to cells. As used herein, the term “electroporation” refers to the use of a transmembrane electric field pulse to induce microscopic pathways (pores) in a bio-membrane. During in vivo electroporation, electrical fields of appropriate magnitude and duration are applied to cells, inducing a transient state of enhanced cell membrane permeability, thus enabling the cellular uptake of molecules unable to cross cell membranes on their own. Creation of such pores by electroporation facilitates passage of biomolecules, such as plasmids, oligonucleotides, siRNAs, drugs, etc., from one side of a cellular membrane to the other. In vivo electroporation for the delivery of DNA vaccines has been shown to significantly increase plasmid uptake by host cells, while also leading to mild-to-moderate inflammation at the injection site. As a result, transfection efficiency and immune response are significantly improved (e.g., up to 1,000 fold and 100 fold respectively) with intradermal or intramuscular electroporation, in comparison to conventional injection.

In a typical embodiment, electroporation is combined with intramuscular injection. However, it is also possible to combine electroporation with other forms of parenteral administration, e.g., intradermal injection, subcutaneous injection, etc.

Administration of a composition or combination of the invention via electroporation can be accomplished using electroporation devices that can be configured to deliver to a desired tissue of a mammal a pulse of energy effective to cause reversible pores to form in cell membranes. The electroporation device can include an electroporation component and an electrode assembly or handle assembly. The electroporation component can include one or more of the following components of electroporation devices: controller, current waveform generator, impedance tester, waveform logger, input element, status reporting element, communication port, memory component, power source, and power switch. Electroporation can be accomplished using an in vivo electroporation device. Examples of electroporation devices and electroporation methods that can facilitate delivery of compositions and combinations of the invention, particularly those comprising DNA plasmids, include CELLECTRA® (Inovio Pharmaceuticals, Blue Bell, PA), Elgen electroporator (Inovio Pharmaceuticals, Inc.) Tri-Grid™ delivery system (Ichor Medical Systems, Inc., San Diego, CA 92121) and those described in U.S. Pat. Nos. 7,664,545, 8,209,006, 9,452,285, 5,273,525, 6,110,161, 6,261,281, 6,958,060, and 6,939,862, 7,328,064, 6,041,252, 5,873,849, 6,278,895, 6,319,901, 6,912,417, 8,187,249, 9,364,664, 9,802,035, 6,117,660, and International Patent Application Publication WO2017172838, the relevant content on electroporation devices and electroporation methods from each of which is herein incorporated by reference in its entirety.

Other examples of in vivo electroporation devices are described in International Patent Application entitled “Method and Apparatus for the Delivery of Hepatitis B Virus (HBV) Vaccines,” filed on the same day as this application with the Attorney Docket Number 688097-405WO1, the contents of which are hereby incorporated by reference in their entireties. Also contemplated by the application for delivery of the compositions and immunogenic combinations of the application are use of a pulsed electric field, for instance as described in, e.g., U.S. Pat. No. 6,697,669, which is herein incorporated by reference in its entirety.

In other embodiments of the application in which a composition or immunogenic combination comprises one or more DNA plasmids, the method of administration is transdermal. Transdermal administration can be combined with epidermal skin abrasion to facilitate delivery of the DNA plasmids to cells. For example, a dermatological patch can be used for epidermal skin abrasion. Upon removal of the dermatological patch, the composition or immunogenic combination can be deposited on the abraised skin.

Methods of delivery are not limited to those described above, and any means for intracellular delivery can be used. Other methods of intracellular delivery contemplated by the methods of the invention include, but are not limited to, liposome encapsulation, nanoparticles, etc.

In certain embodiments of the application, the method of administration is a lipid composition, such as a lipid nanoparticle (LNP). Lipid compositions, preferably lipid nanoparticles, that can be used to deliver a therapeutic product (such as one or more nucleic acid molecules of the invention), include, but are not limited to, liposomes or lipid vesicles, wherein an aqueous volume is encapsulated by amphipathic lipid bilayers, or wherein the lipids coat an interior that comprises a therapeutic product; or lipid aggregates or micelles, wherein the lipid-encapsulated therapeutic product is contained within a relatively disordered lipid mixture.

In particular embodiments, the LNPs comprise a cationic lipid to encapsulate and/or enhance the delivery of a nucleic acid molecule, such as a DNA or RNA molecule of the invention, into the target cell. The cationic lipid can be any lipid species that carries a net positive charge at a selected pH, such as physiological pH. The lipid nanoparticles can be prepared by including multi-component lipid mixtures of varying ratios employing one or more cationic lipids, non-cationic lipids and polyethylene glycol (PEG)—modified lipids. Several cationic lipids have been described in the literature, many of which are commercially available. For example, suitable cationic lipids for use in the compositions and methods of the invention include 1,2-dioleoyl-3-trimethylammonium-propane (DOTAP).

The LNP formulations can include anionic lipids. The anionic lipids can be any lipid species that carries a net negative charge at a selected pH, such as physiological pH. The anionic lipids, when combined with cationic lipids, are used to reduce the overall surface charge of LNPs and to introduce pH-dependent disruption of the LNP bilayer structure, facilitating nucleotide release. Several anionic lipids have been described in the literature, many of which are commercially available. For example, suitable anionic lipids for use in the compositions and methods of the invention include 1,2-dioleoyl-sn-glycero-3-phosphoethanolamine (DOPE).

LNPs can be prepared using methods well known in the art in view of the present disclosure. For example, the LNPs can be prepared using ethanol injection or dilution, thin film hydration, freeze-thaw, French press or membrane extrusion, diafiltration, sonication, detergent dialysis, ether infusion, and reverse phase evaporation.

Some examples of lipids, lipid compositions, and methods to create lipid carriers for delivering active nucleic acid molecules, such as those of this invention, are described in: US2017/0190661, US2006/0008910, US2015/0064242, US2005/0064595, WO/2019/036030, US2019/0022247, WO/2019/036028, WO/2019/036008, WO/2019/036000, US2016/0376224, US2017/0119904, WO/2018/200943, WO/2018/191657, US2014/0255472, and US2013/0195968, the relevant content of each of which is hereby incorporated by reference in its entirety.

### Kits

Also provided herein is a kit comprising a pharmaceutical combination of the application and instructions for use. A kit can comprise the fusion protein and immunogen in separate compositions, or a kit can comprise the fusion protein and immunogen in a single composition.

The ability to induce or stimulate an immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [3H]thymidine uptake or flow cytometry-based assays), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

### Embodiments

The invention provides also the following non-limiting embodiments.

Embodiment 1 is a fusion protein comprising a) an IL12 p40 subunit; b) a linker consisting of the amino acid sequence of SEQ ID NO: 3; and c) an IL12 p35 subunit; wherein the fusion protein is arranged from N-terminus to C-terminus in the order (a)-(b)-(c), and the C-terminus of the IL12 p40 subunit is fused to the N-terminus of the IL12 p35 subunit through the linker.

Embodiment 1a is a fusion protein comprising a) an IL12 p40 subunit; b) a linker consisting of the amino acid sequence of SEQ ID NO: 3; and c) an IL12 p35 subunit; wherein the fusion protein is arranged from N-terminus to C-terminus in the order (c)-(b)-(a), and the C-terminus of the IL12 p35 subunit is fused to the N-terminus of the IL12 p40 subunit through the linker.

Embodiment 2 is the fusion protein of embodiment 1 or 1a, wherein the p40 subunit comprises an amino acid sequence having at least 90% sequence identity to SEQ ID NO: 1, 7, or 9.

Embodiment 3 is the fusion protein of embodiment 1 or 1a, wherein the p35 subunit comprises an amino acid sequence having at least 90% sequence identity to SEQ ID NO: 2, 8, or 10.

Embodiment 4 is the fusion protein of embodiment 1, comprising an amino acid sequence having at least 90% sequence identity to SEQ ID NO: 24.

Embodiment 5 is the fusion protein of embodiment 1, comprising an amino acid sequence having at least 90% identity to SEQ ID NO: 25.

Embodiment 6 is the fusion protein of embodiment 1, comprising an amino acid sequence having at least 90% sequence identity to SEQ ID NO: 26.

Embodiment 7 is a fusion protein comprising the amino acid sequence of SEQ ID NO: 24.

Embodiment 8 is the fusion protein of any one of embodiments 1-7, further comprising a signal sequence operably linked to the N-terminus of the p40 subunit, preferably, the signal sequence is selected from the group consisting of SEQ ID NOs: 11, 12, and 13.

Embodiment 9 is an isolated nucleic acid molecule comprising a nucleotide sequence encoding the fusion protein of any one of embodiments 1-8.

Embodiment 10 is the isolated nucleic acid molecule of embodiment 9, having at least 90% sequence identity to SEQ ID NOs: 27, 28, or 29.

Embodiment 11 is the isolated nucleic acid molecule comprising the nucleotide sequence of SEQ ID NO: 27.

Embodiment 12 is a vector comprising the nucleic acid molecule of any one of embodiments 9 to 11.

Embodiment 12a is the vector of embodiment 12, wherein the vector is a DNA vector.

Embodiment 12b is the vector of embodiment 12a, wherein the DNA vector is selected from the group consisting of DNA plasmids, bacterial artificial chromosomes, yeast artificial chromosomes, and closed linear deoxyribonucleic acid.

Embodiment 12c is the vector of embodiment 12, wherein the vector is an RNA vector.

Embodiment 12d is the vector of embodiment 12c, wherein the RNA vector is an RNA replicon, preferably a self-replicating RNA replicon, an mRNA replicon, a modified mRNA replicon, or self-amplifying mRNA.

Embodiment 12e is the vector of embodiment 12, wherein the vector is a viral vector.

Embodiment 12f is the vector of embodiment 12e, wherein the viral vector is selected from the group consisting of bacteriophages, animal viruses, and plant viruses.

Embodiment 12g is the vector of embodiment 12, wherein the vector is a linear covalently closed double-stranded DNA that is structurally distinct from plasmid DNA.

Embodiment 12h is the vector of embodiment 12g, wherein the vector is “Doggybone™ closed linear DNA” (dbDNA™) (Touchlight Genetics Ltd.; London, England).

Embodiment 13 is a host cell comprising the nucleic acid molecule of any one of embodiments 9 to 11 or the vector of any one of embodiments 12 to 12h.

Embodiment 14 is a pharmaceutical composition comprising the fusion protein of any one of embodiments 1 to 7 and a pharmaceutically acceptable carrier.

Embodiment 15 is a pharmaceutical composition comprising the nucleic acid molecule of any one of embodiments 9 to 11 or the vector of any one of embodiments 12 to 12 h and a pharmaceutically acceptable carrier.

Embodiment 16 is a kit or a pharmaceutical combination comprising the fusion protein of any one of embodiments 1 to 7, the nucleic acid molecule of any one of embodiments 9-11 or the vector of any one of embodiments 12 to 12h, and an immunogen.

Embodiment 17 is a kit or pharmaceutical combination of embodiment 16, wherein the immunogen is capable of inducing an immune response against an infectious agent, or a disease.

Embodiment 18 is a method of enhancing an immune response in a subject in need thereof, comprising administering to the subject an effective amount of the fusion protein of any one of embodiments 1-7, the nucleic acid molecule of any one of embodiments 9-11 or the vector of any one of embodiments 12-12h.

Embodiment 19 is a method of inducing an immune response in a subject in need thereof, comprising administering to the subject an immunologically effective amount of an immunogen and at least one of the fusion proteins of any one of embodiments 1-7, the nucleic acid molecule of any one of embodiments 9-11 and the vector of any one of embodiments 12 to 12h.

Embodiment 19a is the method of embodiment 19, wherein the immunologically effective amount of the immunogen is administered in combination with the at least one of the fusion proteins of any one of embodiments 1-7, the nucleic acid molecule of any one of embodiments 9-11 and the vector of any one of embodiments 12 to 12h.

Embodiment 19b is the method of embodiment 19 or 19a, wherein the immunogen is capable of inducing an immune response against an infectious agent or disease.

Embodiment 19c is the method of embodiment 19b, wherein the immunogen is capable of inducing an immune response against an infectious agent, such as a virus (e.g., human immunodeficiency virus (HV), influenza, respiratory syncytial virus (RSV), Ebola virus, hepatitis B virus HBV), hepatitis C virus (HCV), human papilloma virus (HPV), Epstein-Barr virus, yellow fever virus, rubella virus, varicella zoster virus, variola virus, mumps virus, measles virus, herpes virus and vaccinia virus) or a pathogen (e.g. a bacterial, parasitic, or fungal pathogen).

Embodiment 19d is the method of embodiment 19b, wherein the immunogen is capable of inducing an immune response against a disease, such as an oncogenic disease (e.g., melanoma, lung cancer, squamous carcinomas of the lung, head and neck cancer, breast cancer, ovarian cancer, uterine cancer, prostate cancer, gastric carcinoma, cervical cancer, esophageal, carcinoma, bladder cancer, kidney cancer, brain cancer, liver cancer, colon cancer, bone cancer, pancreatic cancer, skin cancer, cutaneous or intraocular malignant melanoma, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, esophagus cancer, small intestine cancer, endocrine system cancer, thyroid gland cancer, parathyroid gland cancer, adrenal gland cancer, sarcoma of soft tissue, urethra cancer, penis cancer, chronic or acute leukemias including acute myeloid leukemia, chronic myeloid leukemia, acute lymphoblastic leukemia, chronic lymphocytic leukemia, solid tumors of childhood, lymphocytic lymphoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, and T-cell lymphoma).

Embodiment 19e is the method of any one of embodiments 18-19d, wherein a composition or combination comprising at least one of the fusion proteins of any one of embodiments 1-7, the nucleic acid molecule of any one of embodiments 9-11 and the vector of any one of embodiments 12 to 12h is administered by injection through the skin, e.g., intramuscular or intradermal injection, preferably intramuscular injection.

Embodiment 19f is the method of embodiment 19e, wherein the composition or combination comprising the nucleic acid molecule of any one of embodiments 9-11 or the vector of any one of embodiments 12 to 12h is administered by intramuscular injection in combination with electroporation.

Embodiment 19g is the method of any one of embodiments 18-19d, wherein a composition or combination comprising the nucleic acid molecule of any one of embodiments 9-11 or the vector of any one of embodiments 12 to 12h is administered to the subject by a lipid composition, preferably by a lipid nanoparticle.

Embodiment 20 is the fusion protein of any one of embodiments 1-7, the nucleic acid molecule of any one of embodiments 9-11 or the vector of any one of embodiments 12-12h for use in enhancing an immune response in a subject in need thereof.

Embodiment 21 is an immunogen and at least one of the fusion proteins of any one of embodiments 1-7, the nucleic acid molecule of any one of embodiments 9-11 and the vector of any one of embodiments 12-12h for use in inducing an immune response in a subject in need thereof.

Embodiment 22 is the method of any one of embodiments 18 and 19, wherein the fusion protein of any one of embodiments 1-7, the nucleic acid molecule of any one of embodiments 9-11 or the vector of any one of embodiments 12-12h is administered by intramuscular injection.

Embodiment 23 is the kit of any one of embodiments 16 or 17 and instructions for use.

Embodiment 24 are products containing the immunogenic combination or kit of claim 16 or 17 as a combined preparation for simultaneous, separate or sequential use in enhancing an immune response induced by the immunogen, in a subject in need thereof.

### EXAMPLES

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example 1. Generation of pcDNA-P40-KE-P35 Construct

Preparation of IL12 p35 and p40 subunit plasmids A pcDNA-P40-KE-P35 construct was generated by first preparing two separate DNA plasmids, containing the human IL12 p40 subunit (SEQ ID NO: 1) and human IL12 p35 subunit (SEQ ID NO: 2), respectively, using standard molecular cloning procedures. Briefly, gBlocks® Gene Fragments were ordered from Integrated DNA Technologies (IDT; Coralville, IA) and used in the following PCR reaction mixtures to amplify the subunits:

### PCR Mixture 1:

PCR mastermix (dNTP, MgCl₂, Taq polymerase)
Nhe-p40 primer (SEQ ID NO: 14)
Xho-p40 primer (SEQ ID NO: 15)
gBlock DNA
H₂O

### PCR Mixture 2:

PCR mastermix (dNTP, MgCl₂, Taq polymerase)
Nhe-P35 primer (SEQ ID NO: 16)
Xho-P35 primer (SEQ ID NO: 17)
gBlock DNA
H₂O

The PCR products were double digested with Nhe and Xho enzymes (Fermentas; Waltham, MA) by adding 1 μl of each enzyme and 3.4 μl of 10× FastDigest buffer (Catalog #B64; Thermo Scientific; Waltham, MA) to the PCR product and incubating at 37° C. for 30 minutes.

Next, the pcDNA3.1 backbone (Thermo Fisher Scientific) was double digested with Nhe and Xho enzymes in 10× FastDigest buffer and H₂O. The reaction was incubated at 37° C. for 60 minutes. Then 1 μl FastAp Thermosensitive Alkaline Phosphatase (Catalog #EF0564; Thermo Scientific) was added to the reaction and incubated for 10 minutes at 37° C. The enzymes were then inactivated by incubating the reaction mixture for 10 minutes at 80° C. The digested vectors were then run on an agarose gel at 100V for a minimum 30 minutes with the total time depending on the size of fragment/vectors. The digested DNA was then cut from the gel and purified according to standard techniques.

The p35 and p40 DNA fragments were each ligated into a digested pcDNA3.1 backbone using Quick Ligation™ kit (Catalog #M2200L; NEB) to generate pcDNA-p35 and pcDNA-p40 plasmids.

### Fusion of p35 and p40 Plasmids

To generate the p40-KE-p35 fusion plasmid, an intermediate plasmid with a TC linker was first generated. The plasmid was generated using standard molecular cloning techniques as described above for the generation of the pcDNA-p40 and pcDNA-p35 plasmids. Briefly, PCR reactions were carried out on a p40-TC-p35 gBlock DNA fragment using Apa-p40 and Xho-EcoRV-p35 primers. Next, the Apa-p40-TC PCR product and the pcDNA-p40 plasmid were double digested with Apa and Xho enzymes. After DNA purification, the Apa-p40-TC fragment was ligated into the cut pcDNA-p40 plasmid to generate pcDNA-P40-TC plasmid. Next, EcoRV-p35-Pme fragments were generated by PCR on the pcDNA-p35 plasmid using EcoRV-p35 and Pme-p35 primers. The EcoRV-p35-Pme PCR product and pcDNA-p40-TC plasmid were then double digested with EcoRV and Pme. After DNA purification, the EcoRV-p35-Pme fragment was ligated into the pcDNA-p40-TC plasmid to generate pcDNA-p40-TC-p35 fusion plasmid.

Next, PCR was carried out on a p40-KE-p35 gBlock DNA fragment using Apa-p40 and Pme-Xho-EcoRV-p35 primers. The Apa-p40-KE-p35-EcoRV PCR product and pcDNA-p40-TC-p35 plasmid were then double digested with Apa and EcoRV enzymes. After DNA purification, the p40-KE-p35 fragment was ligated into the cut pcDNA-p40-TC-p35 plasmid to generate the pcDNA-p40-KE-p35 fusion plasmid (FIG. 1).

Similar molecular cloning methods to those described above were used to generate pDK-p40-KE-p35 fusion construct. This construct contains a kanamycin resistance cassette which replaces the ampicillin resistance cassette of the pcDNA plasmid, making it suitable for use in vivo (FIG. 2).

In addition to the KE linker (SEQ ID NO: 3), plasmids were generated using other another linker, and/or to include one or more additional elements, such as the coding sequences for RNA-binding protein 3 IRES (IR; SEQ ID NO: 4) and FMDV (Foot Mouth Disease Virus) 2A peptide (FA; SEQ ID NO: 5) and an overlapping stop and start codon, termed translational coupling spacer (TC; SEQ ID NO: 6), in a similar manner as described above.

### Example 2. IL12 Protein Production and Secretion

To confirm that recombinant IL12 p70 protein was expressed from the pcDNA-P40-KE-P35 construct, the pcDNA-KE-P35 construct was transfected into human embryonic kidney (HEK) 293T (ATCC 11268) cells. Prior to transfection, cells were grown as adherent cultures. The transfections were performed according to standard procedures using PEI transfection reagent (Polyplus-transfection; Illkirch-Graffenstaden, France).

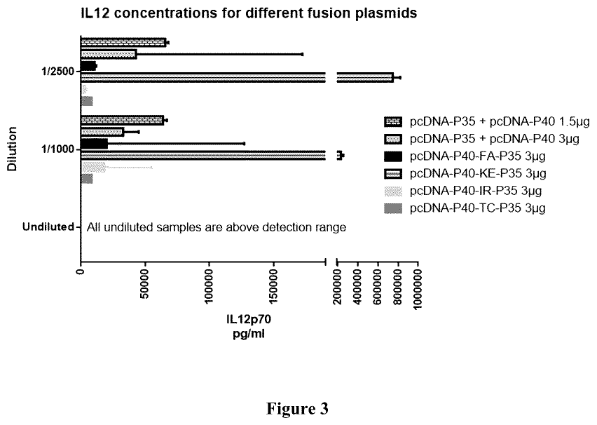

The second day after transfection, the cell medium was collected and centrifuged for 5 minutes at 1500 rpm to remove cells/cell debris. The supernatant was stored at −20° C. or used immediately for a IL12 p70 ELISA. Next, the Human IL-12 p70 Quantikine ELISA Kit (R&D Systems Catalog #D1200; Minneapolis, MN) protocol was performed according to the kit's manual. The concentration of the IL12 p70 secreted protein from cells transfected with pcDNA-P40-FA-P35, pcDNA-p40-KE-p35, pcDNA-p40-IR-p35, pcDNA-p40-TC-p35, or pcDNA-p35 and pcDNA-p40 was determined by comparison to a standard curve of known IL12 p70 concentrations. FIG. 3 shows that transfection of cells with pcDNA-p40-KE-p35 fusion plasmid resulted in the highest IL12 p70 compared to fusion plasmids with other linkers.

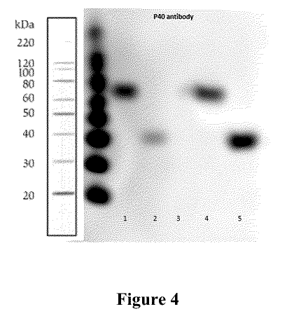

Western Blot analysis was used to assess whether the secreted recombinant IL12 p70 protein was maintained as a fused IL12 p70 protein or separated into IL12 p40 and IL12 p35 protein subunits. Cell lysates and supernatant were collected from cells transfected with the pDK-p40-KE-p35 construct and cells transfected with both the pcDNA-p35 and the pcDNA-p40 constructs. IL12 p40 expression was assessed in cell lysates and supernatant by sodium dodecyl sulfatepolyacrylamide denaturing gel electrophoresis (SDS-PAGE) followed by detection using an anti-IL12 p40 antibody (Thermo Scientific Catalog #701233). FIG. 4 shows that for pDK-p40-KE-p35, the IL12 protein only results in a p40 subunit that is maintained in the IL12 p70 heterodimeric fusion protein.

### Example 3. Biological Activity of Secreted IL12p70 Protein

IL12 is a proinflammatory cytokine that induces Interferon gamma (IFNg) production by T cells. To assess whether the p40-KE-p35 fusion protein expressed from the plasmid functioned as an IL12 p70 protein, the fusion protein's ability to stimulate CD3 T cells to produce IFNg was tested. CD3 T cells were isolated from two human donor samples (DN921 and DN922) using CD3 MicroBeads (MiltenyiBiotec; Bergisch Gladbach, Germany) according to manufacturer's instructions. CD3 T cells were maintained in IMDM Iscove's Modified Dulbecco's medium with 20% FBS. CD3 T cells were stimulated with anti-CD3 antibody (BD Biosciences Catalog #555336; Franklin Lakes, New Jersey), anti-CD28 antibody (Sanquin Catalog #M1650; Amsterdam, The Netherlands), and supernatant containing IL12 p70. Supernatants from cells transfected with the pcDNA-p40-KE-p35 construct and cells transfected with both the pcDNA-P35 and the pcDNA-P40 constructs were tested for biological activity. Recombinant human IL12 p70 (Peprotech; Rocky Hill, NJ) was used as a positive control. Prior to stimulation, concentrations of IL12 p70 in the supernatants were first measured by ELISA as described above in order to ensure equal concentrations of IL12 p70 from the supernatant and the recombinant IL12 p70 were used to stimulate the CD3 T cells. After incubating the CD3 T cells in stimulation media for 3 days at 37° C., the culture plates were spun down and supernatant was collected. Supernatant was either frozen at −20° C. or immediately tested for IFNg concentration.

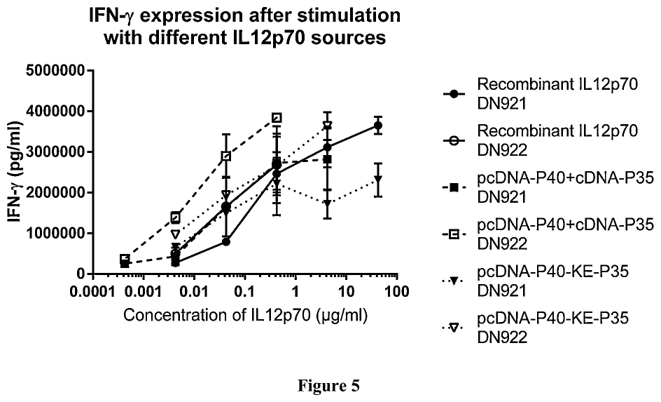

IFNg concentrations were measured using the V-PLEX NHP IFN-γ Kit (Meso Scale Discovery Catalog #K156QOD; Rockville, Maryland). IL12 p70 expressed from the pcDNA-p40-KE-p35 construct induced CD3 T cells to produce IFNg comparable to the recombinant IL12p70 positive control. Increasing the concentration of IL12p70 led to a corresponding increase in IFNg production (FIG. 5). These results confirmed that the p40-KE-p35 fusion protein had the functional activity of an IL12 p70 protein.

### Example 4. In Vivo Immune Stimulation with p40-KE-p35 Construct

This example describes experiments testing whether a plasmid encoding the p40-KE-p35 fusion protein can enhance T cell responses to Hepatitis B Virus (HBV) core (HBc) and HBV viral polymerase (Pol) antigens in mice. The HBV core protein is the subunit of the viral nucleocapsid. Pol is needed for synthesis of viral DNA (reverse transcriptase, RNaseH, and primer), which takes place in nucleocapsids localized to the cytoplasm of infected hepatocytes. Both the HBV core and Pol are antigens capable of inducing an immune response to HBV.

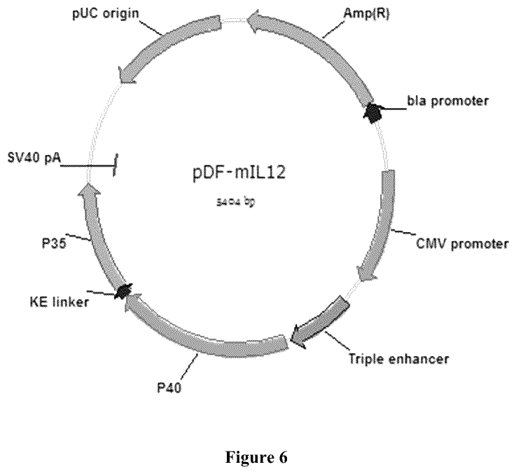

For these experiments, a mouse p40-KE-p35 fusion plasmid was first constructed. Plasmids containing mouse IL12 p40 (SEQ ID NO: 7) and p35 (SEQ ID NO: 8) subunits were generated as described in Example 1 using gBlock® MP40KEP35 (IDT) with Eco and Xho restriction sites. The mouse p40-KE-p35 fusion construct was then cloned into a pDF vector using standard molecular cloning techniques as described in Example 1 (FIG. 6). The pDF vector contains an ampicillin resistance cassette in place of the kanamycin resistance cassette.

### Immunization

The following plasmids were used in the experiment: pDF-HBV core, pDF-HBV pol, pDF-p40-KE-p35, pUMVC3 mIL12-IRES (Ichor), pDK empty vector. The pDF-HBV core and pDF-HBV pol are described in U.S. patent application Ser. No. 16/223,251, filed Dec. 18, 2018, the contents of which is hereby incorporated by reference in their entireties. The pUMVC3 mIL12-IRES plasmid was provided by Ichor Medical Systems (San Diego, CA). The pUMVC3 mIL12-IRES construct is a bicistronic construct whereby p35 and p40 are linked together on the nucleotide level with an EMCV IRES sequence, thus the plasmid makes the p35 and p40 proteins separately.

The DNA plasmid (pDNA) vaccine along with various amounts of the IL-12-expressing plasmids was intramuscularly delivered via electroporation to Balb/c mice using a commercially available TriGrid™ delivery system-intramuscular (TDS-IM) (Ichor Medical Systems) adapted for application in the mouse model in cranialis tibialis. Forty-four female BALB/c mice, 8-9 weeks old, were injected with a combination of plasmids as outlined in Table 1. Six mice were administered plasmid DNA encoding the HBV core antigen and HBV pol antigen (pDF-core+pDF-pol; Group 1), groups of six mice were administered plasmid DNA encoding the HBV core antigen and HBV pol antigen with 0.1 μg, 0.5 μg, 2.0 μg of plasmid DNA encoding p40-KE-p35 fusion, respectively (pDF-core+pDF-pol+pDF-p40-KE-p35; Groups 2, 3, 4); groups of six mice were administered plasmid DNA encoding the HBV core antigen and HBV pol antigen with 0.1 μg, 0.5 μg, 2.0 μg of plasmid DNA encoding Ichor's mIL12, respectively (pDF-core+pDF-pol+pUMVC3 mIL12-IRES; Group 5, 6, and 7), and two mice received empty vector as the negative control (pDK-empty; Group 8). Animals received two DNA immunizations three weeks apart and splenocytes were collected one week after the last immunization.

TABLE 1

| | | | | | | Unilateral | | |
|---|---|---|---|---|---|---|---|---|
| | | | | | | Admin | | |
| | | | | | | Site | | Endpoint |
| | | | pDNA | Total | Total | (Alternate | Admin | (spleen |
| Group | N | DNA | Dose | Vol | (pDNA) | sides) | Days | harvest) |
| 1 | 6 | pDF-core | 0.5 μg | 20 μl | 0.05 mg/ml | CT + EP | 0, 21 | D 28 |
| | | pDF-pol | 0.5 μg | | | | | |
| 2 | 6 | pDF-core | 0.5 μg | 20 μl | 0.055 mg/ml | CT + EP | 0, 21 | D 28 |
| | | pDF-pol | 0.5 μg | | | | | |
| | | pDF-p40-KE-p35 | 0.1 μg | | | | | |
| 3 | 6 | pDF-core | 0.5 μg | 20 μl | 0.075 mg/ml | CT + EP | 0, 21 | D 28 |
| | | pDF-pol | 0.5 μg | | | | | |
| | | pDF-p40-KE-p35 | 0.5 μg | | | | | |
| 4 | 6 | pDF-core | 0.5 μg | 20 μl | 0.15 mg/ml | CT + EP | 0, 21 | D 28 |
| | | pDF-pol | 0.5 μg | | | | | |
| | | pDF-p40-KE-p35 | 2.0 μg | | | | | |
| 5 | 6 | pDF-core | 0.5 μg | 20 μl | 0.055 mg/ml | CT + EP | 0, 21 | D 28 |
| | | pDF-pol | 0.5 μg | | | | | |
| | | pUMVC3 | 0.1 μg | | | | | |
| | | mIL12-IRES | | | | | | |
| 6 | 6 | pDF-core | 0.5 μg | 20 μl | 0.075 mg/ml | CT + EP | 0, 21 | D 28 |
| | | pDF-pol | 0.5 μg | | | | | |
| | | pUMVC3 | 0.5 μg | | | | | |
| | | mIL12-IRES | | | | | | |
| 7 | 6 | PDF-core | 0.5 μg | 20 μl | 0.15 mg/ml | CT + EP | 0, 21 | D 28 |
| | | PDF-pol | 0.5 μg | | | | | |
| | | pUMVC3 | 2.0 μg | | | | | |
| | | mIL12-IRES | | | | | | |
| 8 | 2 | pDK-empty | 1.0 μg | 20 μl | 0.05 mg/ml | CT + EP | 0, 21 | D 28 |

N—number of mice per group;
pDNA—plasmid DNA;
CT—cranial tibialis;
EP—electroporation;
D—days

### T Cell Activity Assay

Antigen-specific responses were analyzed and quantified by IFNg enzyme-linked immunospot (ELISPOT). In this assay, isolated splenocytes of immunized animals were incubated overnight with peptide pools covering the HBV Core protein and the HBV Pol protein. A pool of 35 peptides was used for the HBV Core protein. A pool of 103 peptides was used for the HBV Pol1. A pool of 105 peptides was used for HBV Pol2. Dimethyl sulfoxide (DMSO) was used as a negative control, and Concanavalin A (ConA) was used as a positive control.

Antigen-specific T cells were stimulated with the homologous peptide pools and IFNg-positive T cells were assessed using the ELISPOT assay. IFN-γ release by a single antigen-specific T cell was visualized by appropriate antibodies and subsequent chromogenic detection as a colored spot on the microplate referred to as spot-forming cell (SFC). A spot is formed for every T cell that secretes IFNg which is a marker for T cell activity.

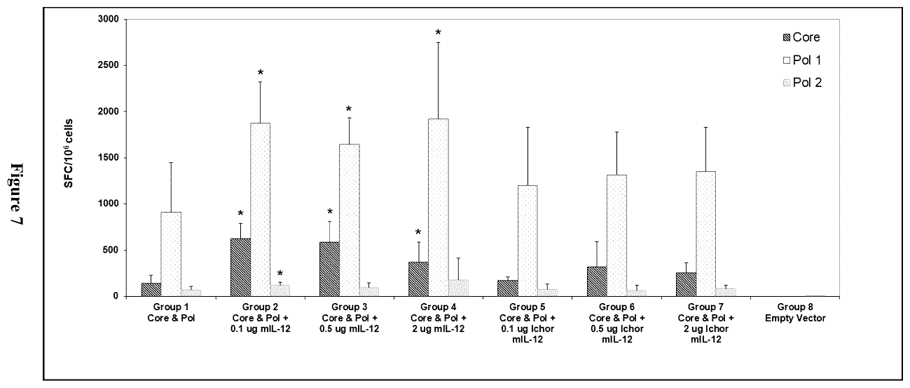

Administration of the pDF-p40-KE-p35 adjuvant in combination with HBV core and pol plasmids resulted in a significantly (p<0.05) higher amount of T cells producing IFNg compared to the Core and Pol plasmids administered without adjuvant (FIG. 7). There was no difference in activity with increasing concentrations of pDF-p40-KE-p35 (0.1 μg/0.5 μg/2 μg). In comparison, administration of the pUMVC3 mIL12-IRES plasmid in combination HBV core and pol plasmids did not lead to a significant increase in T cell activity compared to the HBV Core and Pol plasmids administered alone. These results demonstrate the IL12 p40-KE-p35 fusion protein acts as an adjuvant to enhance immune responses to vaccines in vivo.

The fusion construct according to embodiments of the invention produces equimolar amounts of the two IL-12 subunits p40 and p35. The single fusion protein of the invention ensures heterodimerization of the p40 and p35 domains resulting in predominant, if not exclusive, generation of the biologically active p70 heterodimer. The fusion protein also circumvents the formation of p40 homodimers which would compete with the p70 heterodimer for receptor binding. Results from the animal study described above demonstrate that a fusion construct of the invention resulted in more enhancement of antigen-specific T cell responses than a heterodimer p70 produced via bicistronic coexpression of the p40 and p35 subunits.

1. A fusion protein comprising
a) an IL12 p40 subunit;
b) a linker consisting of the amino acid sequence of SEQ ID NO: 3; and
c) an IL12 p35 subunit;
wherein the fusion protein is arranged from N-terminus to C-terminus in the order (a)-(b)-(c), and the C-terminus of the IL12 p40 subunit is fused to the N-terminus of the IL12 p35 subunit through the linker.

2. The fusion protein of claim 1, wherein the p40 subunit comprises an amino acid sequence having at least 90% sequence identity to SEQ ID NO: 1, 7, or 9.

3. The fusion protein of claim 1 wherein the p35 subunit comprises an amino acid sequence having at least 90% sequence identity to SEQ ID NO: 2, 8, or 10.

4. The fusion protein of claim 1, comprising an amino acid sequence having at least 90% sequence identity to SEQ ID NO: 24.

5. The fusion protein of claim 1, comprising an amino acid sequence having at least 90% identity to SEQ ID NO: 25.

6. The fusion protein of claim 1, comprising an amino acid sequence having at least 90% sequence identity to SEQ ID NO: 26.

7. A fusion protein comprising the amino acid sequence of SEQ ID NO: 24.

8. The fusion protein of claim 1, further comprising a signal sequence operably linked to the N-terminus of the p40 subunit.

9. An isolated nucleic acid molecule comprising a nucleotide sequence encoding the fusion protein of claim 1.

10. The isolated nucleic acid molecule of claim 9, having at least 90% sequence identity to SEQ ID NOs: 27, 28, or 29.

11. The isolated nucleic acid molecule of claim 10, comprising the nucleotide sequence of SEQ ID NO: 27.

12. A vector comprising the nucleic acid molecule of claim 9.

13. A host cell comprising the nucleic acid molecule of claim 9.

14. A pharmaceutical composition comprising the fusion protein of claim 1 and a pharmaceutically acceptable carrier.

15. A pharmaceutical combination comprising the fusion protein of claim 1 and an immunogen.

16. The pharmaceutical combination of claim 15, wherein the immunogen induces an immune response against an infectious agent, or a disease.

17. A method of enhancing an immune response in a subject in need thereof, comprising administering to the subject the fusion protein of claim 1.

18. A method of inducing an immune response in a subject in need thereof, comprising administering to the subject the pharmaceutical combination of claim 15.

19. (canceled)

20. The method of claim 18, wherein the fusion protein and the immunogen are administered simultaneously, separately, or sequentially.

21. The fusion protein of claim 8, wherein the signal sequence is selected from the group consisting of SEQ ID NOs: 11, 12, and 13.


SEQUENCE LISTING (29 sequences)

SEQ ID NO: 1 - protein, 306 aa, Artificial Sequence
  human p40 subunit
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 197 aa, Artificial Sequence
  human p35 subunit
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 18 aa, Artificial Sequence
  KE linker
KESGSVSSEQLAQFRSLD

SEQ ID NO: 4 - protein, 7 aa, Artificial Sequence
  RNA-binding protein 3 IRES
FIISSSR

SEQ ID NO: 5 - protein, 22 aa, Artificial Sequence
  FMDV 2A peptide
VKQTLNFDLLKLAGDVESNPGP

SEQ ID NO: 6 - DNA, 5 bp, Artificial Sequence
  translational coupling spacer
tgatg

SEQ ID NO: 7 - protein, 313 aa, Artificial Sequence
  Mouse p40 subunit
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 8 - protein, 193 aa, Artificial Sequence
  mouse p35 subunit
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 9 - protein, 306 aa, Artificial Sequence
  cynomolgus p40 subunit
IWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQVEVSWEYPDTWSTPHSYFSLTFCIQVQGKSKREKKDRIFTDKTSATVICRKNASFSVQAQDRYYSSSWSEWASVPCS

SEQ ID NO: 10 - protein, 252 aa, Artificial Sequence
  cynomolgus p35 subunit
WPPGSASQPPPSPATATGLHPAARPEPLQCRLSMCPARSLLLVATLVLLDYLSLARNLSVATPGPEMFPCLHHSQNLLKAASNTLQKARQILEFYPCTSEEIDHEDITKDKTSTVDACLPLELIKNESCLNSRETSFITNGSCLASRKTSFMMALCLRSIYEDLKMYQVEFKTMNAKLLRDPKRQIFLDQNILGVIDELMQALNFNSETVPQKSSLEEPDFYKTKIKLCILLHAFRIRAVTIDRVMSYLNAS

SEQ ID NO: 11 - protein, 22 aa, Artificial Sequence
  Human p40 signal peptide
MCHQQLVISWFSLVFLASPLVA

SEQ ID NO: 12 - protein, 22 aa, Artificial Sequence
  Mouse p40 signal peptide
MCPQKLTISWFAIVLLVSPLMA

SEQ ID NO: 13 - protein, 22 aa, Artificial Sequence
  cynomolgus p40 signal peptide
MCHQQLVISWFSLVFLASPLMA

SEQ ID NO: 14 - DNA, 36 bp, Artificial Sequence
  Nhe-p40 oligonucleotide
cctgccgctagcaccatgtgtcaccagcagttggtc

SEQ ID NO: 15 - DNA, 33 bp, Artificial Sequence
  Xho-p40 oligonucleotide
ctggatctcgagctaactgcagggcacagatgc

SEQ ID NO: 16 - DNA, 37 bp, Artificial Sequence
  Nhe-p35 oligonucleotide
gggacagctagcaccatgtggccccctgggtcagcct

SEQ ID NO: 17 - DNA, 37 bp, Artificial Sequence
  Xho-p35 oligonucleotide
ggacctctcgagttaggaagcattcagatagctcatc

SEQ ID NO: 18 - DNA, 584 bp, Artificial Sequence
  hCMV promoter sequence
tgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 19 - DNA, 225 bp, Artificial Sequence
  bGH polyadenylation signal sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 20 - DNA, 99 bp, Artificial Sequence
  Bla promoter sequence
acccctatttgtttatttttctaaatacattcaaatatgtatccgctcatgagacaataaccctgataaatgcttcaataatattgaaaaaggaagagt

SEQ ID NO: 21 - DNA, 671 bp, Artificial Sequence
  pUC ori sequenc
cccgtagaaaagatcaaaggatcttcttgagatcctttttttctgcgcgtaatctgctgcttgcaaacaaaaaaaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 22 - DNA, 795 bp, Artificial Sequence
  Kan R gene
atgattgagcaagatggtcttcacgctggctcgccagctgcgtgggtggaacgcctgtttggttatgattgggcgcagcagactattggatgttccgacgcggctgtatttcggctgtctgctcagggtcgccccgtgctgtttgtgaagacggatttgtctggcgcattaaatgagttacaggacgaggcggctcgtctgagttggttggccaccaccggcgtgccctgcgccgcagtgctggatgtcgtgacagaagcaggccgcgattggctccttctcggcgaagtgccgggccaggacctgctcagcagccacttggcaccggcagaaaaagtttctatcatggccgacgccatgcgtcgtcttcacactctcgatccggccacgtgcccctttgaccaccaggccaagcatcgtattgaacgtgcgcgtactcggatggaagcaggtttagtagaccaggacgatttggatgaggaacatcaaggcctggccccggctgaactgtttgcgcgcttaaaagcgtcgatgccagatggcgaagatttggtagtcacccatggagatgcgtgtttgccaaacatcatggttgaaaatggccgcttctcaggctttattgactgtgggcgcctgggtgttgccgaccgctatcaagatattgcgctcgcaactcgtgacatcgctgaagagctgggcggagaatgggctgaccgtttcctggtactgtatggcattgcagcgcccgattcccaacgcatcgcattttatcgtctgctggatgagtttttctaa

SEQ ID NO: 23 - protein, 264 aa, Artificial Sequence
  Kan R protein
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 24 - protein, 521 aa, Artificial Sequence
  Human p40-KE-p35 fusion protein
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSKESGSVSSEQLAQFRSLDRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - protein, 524 aa, Artificial Sequence
  Mouse p40-KE-p35 fusion protein
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSKESGSVSSEQLAQFRSLDRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 26 - protein, 576 aa, Artificial Sequence
  Cynomolgus p40-KE-p35 fusion protein
IWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQVEVSWEYPDTWSTPHSYFSLTFCIQVQGKSKREKKDRIFTDKTSATVICRKNASFSVQAQDRYYSSSWSEWASVPCSKESGSVSSEQLAQFRSLDWPPGSASQPPPSPATATGLHPAARPEPLQCRLSMCPARSLLLVATLVLLDYLSLARNLSVATPGPEMFPCLHHSQNLLKAASNTLQKARQILEFYPCTSEEIDHEDITKDKTSTVDACLPLELIKNESCLNSRETSFITNGSCLASRKTSFMMALCLRSIYEDLKMYQVEFKTMNAKLLRDPKRQIFLDQNILGVIDELMQALNFNSETVPQKSSLEEPDFYKTKIKLCILLHAFRIRAVTIDRVMSYLNAS

SEQ ID NO: 27 - DNA, 1566 bp, Artificial Sequence
  Human p40-KE-p35 fusion
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtaaagaatctggctcagtaagctcagagcaactggcgcagtttcgtagtctggatagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 28 - DNA, 1575 bp, Artificial Sequence
  Mouse p40-KE-p35 fusion
atgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccaaagaatctggatccgtaagctcagagcaactggcgcagtttcgtagtctggatagggtcattccagtctctggacctgccaggtgtcttagccagtcccgaaacctgctgaagaccacagatgacatggtgaagacggccagagaaaagcttaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaa

SEQ ID NO: 29 - DNA, 1734 bp, Artificial Sequence
  cynomolgus p40-KE-p35 fusion
atctgggagctgaagaaggacgtgtatgttgtggagctcgactggtacccagacgcccctggggaaatggtggtgttgacatgcgacactcccgaagaggatggtattacctggactctggatcagtcaggcgaggttctcggtagcggcaaaaccttgacaattcaagtgaaagagtttggtgatgccggtcagtacacctgtcataagggtggagaggcattgagtcactctttgttgttgctgcataagaaggaagatggaatttggtctactgatgtcctcaaagaccagaaagaaccaaagaacaaaacatttcttcgctgtgaagctaaaaactattccggtcggtttacgtgttggtggctcacaactatctctactgatctgaccttttctgtgaaaagcagccggggttcctccaatccccaaggtgttacatgtggagctgtcaccctgtctgccgagagagtccgcggcgacaataaagaatacgagtattccgtggagtgccaggaagatagcgcatgtcccgcagctgaagagagactccccatcgaggtgatggtggacgccatccacaaactcaaatatgaaaactacacaagcagtttcttcattagggacataattaagcctgatcccccaaagaacctccaactgaaacccttgaaaaactccagacaggtggaggtgagctgggaatatccagacacctggtcaactcctcatagctatttctctctgaccttttgcatccaagtacagggcaagtccaagagggagaagaaagatagaattttcaccgacaaaacgtctgcaacggtcatctgtaggaaaaatgctagtttttccgtacaggctcaggatagatattacagtagcagttggtcagagtgggcaagtgtgccatgttccaaagagagtggatccgttagctccgagcaactcgcccagtttcggtctctagactggcccccaggttctgctagtcagccacctccatcccccgccacagctaccggcctgcaccccgctgctaggcctgagcctctgcagtgtaggctcagtatgtgccccgcacgcagccttctcctcgtggctacactggtactgttggattatctgagtctggcccggaatctgagtgttgctacaccagggcctgagatgtttccatgcctccatcatagtcagaacctgcttaaagccgcctctaataccctgcagaaagctagacaaattctggaattttatccttgcaccagtgaggagattgatcacgaggatattacaaaagataagacctcaactgtcgatgcttgtctgccacttgagttgattaaaaacgagagctgccttaattccagagagactagcttcataactaatggctcctgcctcgccagcaggaagactagttttatgatggcactgtgcctgcggagtatctatgaggatcttaaaatgtatcaggtggagtttaagacaatgaacgccaaactgcttagagatcccaaacgacaaatctttctcgatcagaacattctcggcgtaattgacgagctgatgcaagctctgaattttaattctgagaccgtcccacagaaatcatccctggaagaacctgatttttataaaaccaagatcaagctttgcattctgcttcacgcatttcgcatccgagctgtgacgattgatcgggtgatgtcatacctgaatgcatcttgatag